(19) United States
(12) Patent Application Publication
(10) Pub. No.: US 2007/0051825 A1
(43) Pub. Date: Mar. 8, 2007

(54) VAPOR DISPENSING DEVICE WITH VENT HAVING SELF-REGULATING PRESSURE AND SELF-CLEANING FEATURES

(75) Inventors: Carl Triplett, Scottsdale, AZ (US); Kevin Hafer, Phoenix, AZ (US)

Correspondence Address: SNELL & WILMER, LLP, ONE ARIZONA CENTER, 400 E. VAN BUREN, PHOENIZ, AZ, 85004-2202, US

(73) Assignee: THE DIAL CORPORATION, Scottsdale, AZ (US)

(21) Appl. No.: 11/162,394

(22) Filed: Sep. 8, 2005

(51) Int. Cl. A24F 25/00; A62C 13/62; A61L 9/04

(52) U.S. Cl. 239/34; 239/302; 239/51.5

(57) ABSTRACT

A vapor dispensing device for dispensing vapor into an environment is provided self-regulates vapor pressure of a reservoir with an evaporation chamber and allows lower volatile ingredients to return to the reservoir. In accordance with an exemplary embodiment of the invention, a vent is in communication between an emitter pad in the evaporation chamber of the dispensing device and the reservoir. This vent allows lower volatility ingredients to flow back into the sealed reservoir extending the useful life of the vapor dispensing device.

### FIELD OF INVENTION

The present invention generally relates to vapor dispensing devices and, more particularly, to a vapor dispensing device that self-regulates internal pressure and exhibits enhanced evaporation performance.

### BACKGROUND OF INVENTION

Vapor dispensing products typically include a volatilizable material and a transport system configured to facilitate evaporation of the volatilizable material into the surrounding environment. In some systems, volatilizable material is contained in a reservoir connected by a wick to an evaporation chamber. The chamber may contain emitter pads or other structures that assist in evaporation of the material.

Known vapor dispensing devices of this type may be disadvantaged in a number of respects. For example, as this wicking process progresses, a vacuum is created in the reservoir making the capillary action more difficult. Also, ingredients having lower volatility may accumulate on any structures in the evaporation chamber. As a result, overall evaporation and product performance diminishes as product use lengthens.

Furthermore, while venting a vapor dispensing device for improving performance may be known, one potential disadvantage is containing and re-capturing liquid or condensation that has not evaporated in the evaporation chamber. Another disadvantage is containing material that may have escaped from the reservoir or wicking structures. Previous attempts to solve these problems were not feasible due to product leakage or the need for additional sealing of prior art venting.

Thus, there is a need for a vapor dispensing device that overcomes these and other limitations of the prior art.

### SUMMARY OF INVENTION

While the way that the present invention addresses the disadvantages of the prior art will be discussed in greater detail below, in general, the present invention provides for equalizing the vapor pressure of the volatilizable material reservoir with the evaporation chamber enabling lower volatile ingredients to flow back into the reservoir. This flow flushes and cleans the evaporation structures extending the useful life of vapor dispensing device. By equalizing the pressure of the reservoir with the evaporation chamber, the vapor dispensing device is able to retain the volatilizable material and more efficiently transfer vapor to the environment. Furthermore, by utilizing the evaporation chamber in this manner, the need for additional sealing is obviated thereby maintaining the cost of the vapor dispensing device. As a result, evaporation performance and product life is improved.

### BRIEF DESCRIPTION OF THE DRAWINGS

A more complete understanding of the present invention may be derived by referring to the detailed description and claims when considered in connection with the Figures, where like reference numbers refer to similar elements throughout the Figures, and:

FIG. 1 illustrates a schematic overview of an enhanced vapor dispensing device providing a context in which the present invention can be practiced.

FIG. 2 illustrates a schematic block diagram of an embodiment of the present invention.

FIG. 3 illustrates a schematic block diagram of another embodiment of the present invention.

FIG. 4 illustrates a heat source and emitter pad in accordance with another embodiment of the present invention.

FIG. 5 illustrates an exemplary embodiment of the present invention.

### DETAILED DESCRIPTION

The following description is not intended to limit the scope, applicability or configuration of the invention in any way. Rather, the following description is intended to provide a convenient illustration for implementing various embodiments of the invention. As will become apparent, various changes may be made in the function and arrangement of the elements described in these embodiments without departing from the scope of the invention as set forth in the appended claims. For example, in the context of the present invention, the device described below has particular use in connection with air freshening vaporizer systems. However, generally speaking, various volatilizable materials such as insect repellants, deodorizers, sanitizers, and/or the like are suitable for use in accordance with the present invention.

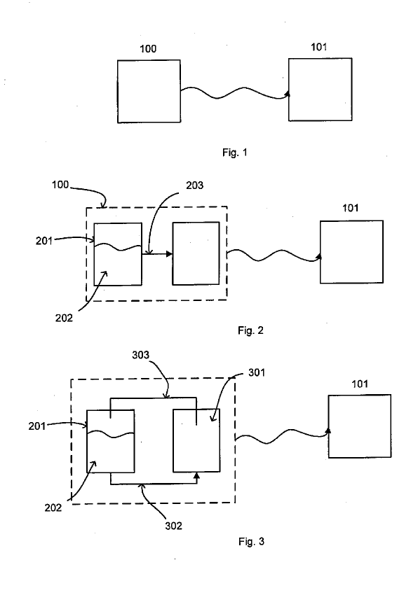

A vapor dispensing device with improved evaporation performance and/or extended product life is provided. This performance is achieved by self-regulating the internal pressure of the device. For example, in an exemplary embodiment of the present invention as shown in FIG. 1, a vapor dispensing device 100 releases vapor from a volatilizable material into the environment 101. The vapor dispensing device comprises any suitable components and configurations that enable the device to communicate the volatized material to the environment.

A vapor dispensing device 100 in accordance with an embodiment of the present invention, as shown in FIG. 2, generally comprises a reservoir 201, filled with volatilizable material 202, connected to a delivery system 203 capable of communication with the environment 101. The reservoir 201 and delivery system 203 generally comprise any components suitably configured to transfer volatilizable material 202 to the environment 101 and volatilizable material 202 generally includes any substance capable of being converted to a vapor. For example, in one exemplary embodiment, volatilizable material 202 is a liquid (e.g., oil, water, or the like scented with various fragrances). As used herein, the environment 101 is any space outside of the vapor dispensing device whether open or enclosed.

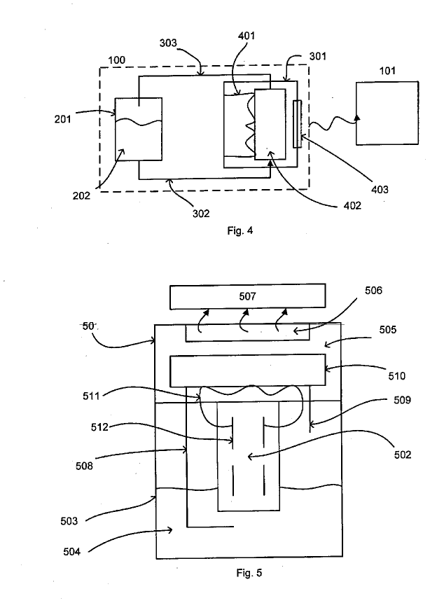

Another embodiment of the present invention, FIG. 3, shows the reservoir 201 may contain one or more compartments encompassing volatilizable material 202 and may be any convenient shape for holding liquid or solid volatilizable material. The delivery system may comprise an evaporation chamber 301, a dispensing structure 302, and a vent 303. The evaporation chamber 301 is in communication with the environment 101 and may contain one or more evaporation devices that facilitate evaporation of volatilizable material 202 into the environment 101. For example, evaporation devices may include an emitter pad or extensions of the dispensing structure 302 into the evaporation chamber 301. The dispensing structure 302 facilitates transfer of volatilizable material 202 to the evaporation chamber 301. The vent 303 connects evaporation chamber 301 to the reservoir 201. The vent 303 in communication with the evaporation chamber 301 equalizes the internal pressure in the reservoir 201 by allowing air from the evaporation chamber 301 to replace volatilizable material 202 that has been delivered to the evaporation chamber 301. Furthermore, the vent 303 in conjunction with the evaporation chamber 301 enables the device to re-capture any volatilizable material that has condensed in the evaporation chamber 301.

In accordance with another embodiment of the invention, the vent 303 may comprise any suitable structure that enables movement of air and volatilizable material 202 that has not evaporated in the evaporation chamber between the reservoir 201 and evaporation chamber 301. The vent 303 may comprise a hole, or preferably a tube. In accordance with various exemplary embodiments, tube is plugged with a porous plastic similar to the dispensing wick providing various potential advantages such as facilitating containment of the volatilizable material during shipment and use.

In another embodiment of the present invention, the evaporation chamber 301 recaptures volatilizable material 202 not evaporated facilitating its return to the reservoir 201. The evaporation chamber 301 may contain one or more evaporation structures such as an emitter pad, wick or suitable structure that enhances transfer of volatilizable material 202 into the environment 101. In accordance with various embodiments of the present invention, the evaporation chamber 301 may be active or passive. For example, in an “active” exemplary embodiment, evaporation chamber 301 may coupled with a heat source 401 that further assists evaporation of volatilizable material 202. Other “active” or “passive” embodiments may include fans, controllable vents, adjustable apertures, heaters or any other structures that assist the evaporation and evacuation of vapor from the chamber into the environment.

As noted above, various embodiments of the present invention may employ delivery systems for transferring vapor to the environment. For example, in one embodiment, as shown in FIG. 4, the evaporation chamber 301 contains an emitter pad 402 in communication with the dispensing structure 302 and the environment 101 providing a surface area that enables enhanced transfer of volatilizable material 202 into the environment 101. The evaporation chamber 301 may be any convenient shape. The emitter pad 402 may be any shape (e.g., rectangular, elliptical, square, triangular, or any other arbitrary rectilinear or curvilinear shape), and may be segmented into one or more sections each with its own connection to one or more dispensing structures. In various embodiments, the evaporation chamber 301 may have an opening 403 configured to allow release of vapor into the environment. Various controls may be attached to the opening 403 to regulate the release of the vapor (e.g., fans, adjustable vents). In one exemplary embodiment, the opening is an open-ended slot opposite the dispensing structure.

In a preferable embodiment, the dispensing structure 302 may contain one or more wicks that supply volatilizable material to the evaporation structure. The wick may be made of any suitable material that facilitates drawing the volatilizable material out of the reservoir. Any number of wicks may be used to assist in controlling any attribute of the dispensing system such as, but not limited to, dispensing speed, multiple fragrance delivery, or final evaporation rate.

In accordance with an exemplary embodiment of the invention, a dispensing wick 303 is connected to the evaporation chamber 301 via an emitter pad 402. The dispensing wick 303 may be configured such that it is an extension of the emitter pad 402.

In another exemplary embodiment, shown in FIG. 5, the vapor dispensing device 501 is configured to at least partially surround an outlet structure 502. For example, in this embodiment, the reservoir 503 containing fragrant oil 504 may ‘U’-shaped and connected to the evaporation chamber 505. Alternatively, the reservoir 503 may be shaped in any convenient manner with any number of sides in order to contain the volatilizable material including but not limited to ‘C’-shapes, ‘L’-shapes or spheres. A slot 506 is open to the environment 507 situated opposite the reservoir 503. The reservoir 503 is not open to the evaporation chamber 505 with exception of the dispensing wick 508 and vent 509. The dispensing wick 508 is immersed in the oil 504 and connects to an emitter pad 510 in the evaporation chamber 505. The vent 509, a tube plugged with porous plastic, is connected to the emitter pad 510 at an end opposite the dispensing wick 508 connection. The second end of the vent 509 is in communication with air in the reservoir 503. A heat source 511 couples a power source 512 and the emitter pad 510.

In accordance with another embodiment of the present invention, configuration of the some or all of the above described components may be situated in a refill component that is removeably attached to a vapor dispensing device.

While various embodiments of enhanced vapor dispensing devices have been described herein, it should be appreciated that any device capable of being configured to enable self-regulation of pressure and self cleaning characteristics, as will be described in greater detail below, may be utilized in accordance with the present invention.

Operational details of an embodiment of an enhanced vapor dispensing device are described below. Volatilizable material is wicked up the dispensing structure into the evaporation chamber. As this evaporation occurs, the evaporation chamber in conjunction with the vent allows the device to self-regulate the internal pressure between the evaporation chamber and the reservoir enhancing evaporation performance. This occurs by allowing air from the evaporation chamber to replace the volatilizable material in the reservoir.

Furthermore, in accordance with an exemplary embodiment of an enhanced vapor dispensing device, the volatilizable material is wicked onto an emitter pad in the evaporation chamber. Higher volatility ingredients in the volatilizable material evaporate into the evaporation chamber and eventually into the environment. Lower volatility ingredients travel the length of the pad without evaporating. As a result of the pressure equalization between the reservoir and the evaporation chamber, the flow speed of volatilizable material across the pad increases. This flow enables the device to self clean by flushing the lower volatility ingredients back into the reservoir. Additionally, the flushing action of volatilizable of material across the emitter pad reduces deposits of lower volatility ingredients on the emitter pad. This flushing action extends device life and enhances capillary action thereby increasing evaporation performance.

Evaporation test data comparing the performance of an exemplary embodiment of the present invention and a non-vented vapor dispensing device is provided. Superior volatilizable material delivery is indicated by the increase in weight of the device after a twenty day test at a given power rating.

TABLE 1
Performance Comparison

| | | | Evaporation | |
|---|---|---|---|---|
| | | Power Setting | Weight (g) | % |
| Vent | Fragrance | (Kohm) | After 20 Days | Increase |
| Non-Vented | Fragrance #1 | 25 | 3.70 | |
| Vented | Fragrance #1 | 25 | 4.95 | 34% |
| Non-vented | Fragrance #1 | 20 | 4.62 | |
| Vented | Fragrance #1 | 20 | 6.92 | 50% |
| Non-vented | Fragrance #2 | 10 | 8.79 | |
| Vented | Fragrance #2 | 10 | 10.47 | 19% |

One test run comprises filling two devices with the same volatilizable material and subjecting the material to the same amount of heat, as represented by the power setting. After twenty days of operation, each device is weighed. The device with the greater weight will have retained more material due to the vented device's ability to return un-evaporated ingredients to the reservoir. Greater retention of material enables enhances performance and a prolonged life of the device.

The present invention has been described above with reference to various exemplary embodiments. However, many changes, combinations and modifications may be made to the exemplary embodiments without departing from the scope of the present invention. For example, the various components may be implemented in alternate ways. These alternatives can be suitably selected depending upon the particular application or in consideration of any number of factors associated with the operation of the system. In addition, the techniques described herein may be extended or modified for use with other types of devices. These and other changes or modifications are intended to be included within the scope of the present invention.

What is claimed is:

1. An vapor dispensing device for dispensing vapor into an environment comprising:
a reservoir wherein said reservoir contains volatilizable material;
an evaporation chamber in communication with the environment;
a dispensing structure in communication between said volatilizable liquid in said reservoir and said evaporation chamber; and
a vent mechanism connecting said evaporation chamber and said reservoir.

2. The device of claim 1, further comprising an emitting structure connecting said dispensing structure to said vent mechanism.

3. The device of claim 2, wherein said emitting structure is an emitter pad.

4. The device of claim 1, wherein said vent mechanism comprises a tube.

5. A device as in claim 4, wherein said tube is porous plastic.

6. The device of claim 1, wherein said volatilizable material is a liquid.

7. The device of claim 6, wherein said liquid is oil-based.

8. A vapor dispensing device for dispensing vapor into an environment comprising:
a reservoir wherein said reservoir contains volatilizable material;
an evaporation chamber in communication with the environment having an emitter pad;
a dispensing wick in communication between said volatilizable liquid in said reservoir and said emitter pad; and
a vent mechanism connecting said emitter pad and said reservoir.

9. The device of claim 8, wherein said volatilizable material is a liquid.

10. The device of claim 9, wherein said liquid is oil-based.

11. The device as in claim 8, wherein said vent mechanism comprises a tube.

12. The device as in claim 11, wherein said tube is porous plastic.

13. A vapor dispensing device for dispensing vapor into an environment comprising:
a reservoir wherein said reservoir contains volatilizable material;
an evaporation chamber in communication with the environment;
a dispensing structure in communication between said volatilizable material in said reservoir and said evaporation chamber; and
means for equalizing vapor pressure between said evaporation chamber and said reservoir.

14. The device of claim 13, further comprising an emitting structure within said evaporation chamber.

15. The device of claim 14, wherein said emitting structure is an emitter pad.

16. The device of claim 13, wherein said volatilizable material is a liquid.

17. The device of claim 16, wherein said liquid is oil-based.

18. A refill component for use in connection with a vapor dispensing device comprising:
a reservoir wherein said reservoir contains volatilizable material;
an evaporation chamber in communication with the environment;
a dispensing structure in communication between said volatilizable liquid in said reservoir and said evaporation chamber; and
a vent mechanism connecting said evaporation chamber and said reservoir.

19. The device of claim 18, further comprising an emitting structure within said evaporation chamber.

20. The device of claim 19, wherein said emitting structure is an emitter pad.

21. The device of claim 1 8, wherein said vent mechanism comprises a tube.

22. A device as in claim 21, wherein said tube is porous plastic.

23. The device of claim 18, wherein said volatilizable material is a liquid.

24. The device of claim 23, wherein said liquid is oil-based.
